(19) United States
(12) Patent Application Publication
(10) Pub. No.: US 2010/0081937 A1
(43) Pub. Date: Apr. 1, 2010

(54) SYSTEM AND METHOD FOR PROCESSING A REAL-TIME ULTRASOUND SIGNAL WITHIN A TIME WINDOW

(76) Inventor: James Hamilton, Brighton, MI (US)

Correspondence Address: SCHOX PLC, 500 3rd Street, Suite 515, San Francisco, CA, 94107, US

(21) Appl. No.: 12/565,666

(22) Filed: Sep. 23, 2009

Related U.S. Application Data
(60) Provisional application No. 61/099,488, filed on Sep. 23, 2008.

(51) Int. Cl. A61B 8/00; A61B 5/0402

(52) U.S. Cl. 600/449; 600/509

(57) ABSTRACT

One embodiment is a method for real-time window processing and window definition that includes measuring a defining signal and an instantaneous tissue state signal, dynamically determining a time window from the defining signal, and processing instantaneous tissue state signal captured in the time window. Another embodiment is a system for processing a real-time ultrasound signal within a time window that includes a physiological signal monitor that measures a physiological signal, an ultrasound acquisition device that acquires an ultrasound signal, a window identifier that uses the physiological signal to identify boundary markers of the output of the physiological signal monitor that define a time window, and an ultrasound processor that processes the ultrasound signal within a time window and outputs a time window processed ultrasound signal. The embodiments have applications in the field of ultrasound tissue tracking and numerous other tissue monitoring fields.

### CROSS-REFERENCE TO RELATED APPLICATIONS

This application claims the benefit of U.S. Provisional Application No. 61/099,488, filed on 23 Sep. 2008, which is incorporated in its entirety by this reference.

### STATEMENT REGARDING FEDERALLY SPONSORED RESEARCH OR DEVELOPMENT

This invention was supported by a grant from the National Heart, Lung, and Blood Institute (#5R44HL071379), and the U.S. government may therefore have certain rights in the invention.

### TECHNICAL FIELD

This invention relates generally to the ultrasound field, and more specifically to a new and useful system and method for processing real-time ultrasound signals within a time window in the ultrasound field.

### BACKGROUND AND SUMMARY

Ultrasound speckle tracking provides accurate measurement of tissue deformation and motion. Tissue velocity and strain rate describe the state of tissue dynamics at a given point in time. In contrast, tissue displacement and strain are accumulated (integrated) measurements, describing the mechanical behavior of tissue over a defined period of time. For example, a useful measurement describing cardiac function is peak systolic strain, defined as the accumulated strain (deformation) from end diastole to peak systole. Currently, most ultrasound speckle tracking techniques are not real-time (i.e., speckle tracking is performed with off-line processing) and accumulation windows are defined by the user or through data processing. For real-time processing, however, accumulation windows must be automatically defined and instantly applied to the data, and not defined or performed off-line after the imaging session.

The present invention includes the steps of measuring a defining signal and an instantaneous tissue state signal, dynamically determining a time window from the defining signal, and processing the tissue state signal captured in the time window. The present invention alleviates the need to manually select or set time windows for tissue state data processing, and furthermore the preferred method allows for real-time feedback for a practitioner at the time of data acquisition. When applied to cardiac tissue speckle tracking, instantaneous measurements such as tissue velocity or strain rate can be processed over a relevant time windows to provide valuable clinical information. Such real-time information has previously been unavailable.

### BRIEF DESCRIPTION OF THE FIGURES

FIG. 1 is a flowchart schematic of the preferred method of the invention.

FIG. 2 is an example of an accumulation window defined by an ECG signal for producing strain images.

FIG. 3 is a schematic representation of the preferred system of the invention.

### DESCRIPTION OF THE PREFERRED EMBODIMENTS

The following description of the preferred embodiments of the invention is not intended to limit the invention to these preferred embodiments, but rather to enable any person skilled in the art to make and use this invention.

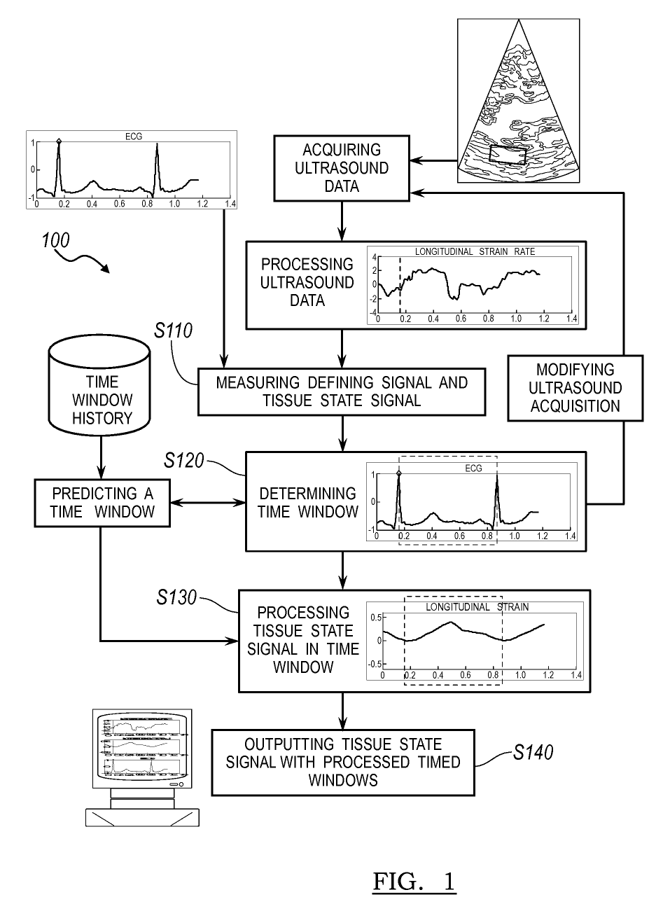

As shown in FIG. 1, a method 100 for processing a real-time ultrasound signal within a time window includes measuring a defining signal and an instantaneous tissue state signal S110, determining a time window from the defining signal S120, and processing the instantaneous tissue state signal captured in the determined time window S130. The ultrasound signal is preferably a image signal but may be any suitable ultrasound signal. For real-time processing of tissue state signals it is advantageous to have accumulation windows automatically defined and applied to the data processing path. The method is preferably applied to speckle tracking in cardiac ultrasound monitoring, but could also be applied to other suitable applications such as measuring of respiratory functions.

Step S110, which includes measuring a defining signal and an instantaneous tissue state signal, functions to measure at least two signals in real time. The defining signal is preferably a physiological signal corresponding to heart electrical activity (e.g., electrocardiogram or ECG), heart rate, blood pressure, respiratory activity, respiration rate, brain waves, reflex reactions, perspiration rate, or any other suitable physiological signal. The physiological signal may be inferred from another signal, such as a respiratory rate may be inferred from heart rate. The defining signal (i.e., the physiological signal) is preferably a non-invasive measurement of a reoccurring event. Preferably, the tissue state signal is an ultrasound signal or processed ultrasound signal. The tissue state signal data is preferably related to the tissue of interest. The tissue state signal is preferably calculated from raw data such as radio-frequency (RF) data from an ultrasound device, but may alternatively be measured data of any tissue. Speckle tracking processing is preferably performed on raw ultrasound data to determine tissue motion. The tissue state signal is preferably an instantaneous measurement of tissue state, such as motion or deformation described by strain rate or tissue velocity. Alternatively, the tissue state signal may be defined as tissue state, blood state, tissue velocity, tissue strain, tissue strain rate, tissue displacement, blood velocity, blood turbulence, or any other suitable tissue state signal. The tissue state signal may additionally be a high frame rate data set. Frame rates of at least 100 frames per second are preferably used to adequately capture the tissue motion of the heart. Furthermore, the defining signal may be a tissue state signal or a signal derived from a tissue state signal. Preferably, the time window has timing aspects that relate to the timing of events of the tissue state signal. As exemplified in FIG. 2, the instantaneous tissue state signal may be strain rate measurements, represented by the image of the upper right panel. Temporal variation of strain rate at the region of interest (ROI) indicated by the box may be presented as a strain rate vs. time plot (upper left panel). The defining signal, defining the accumulation window, may be presented as a conventional electrocardiogram (ECG) signal (lower left panel). From the instantaneous tissue state (e.g., strain rate) and defining signal (e.g., ECG), the accumulated tissue state is calculated, in this case strain measurements (lower right panel). Similar to strain rate, the temporal variation of strain at the ROI may be presented as a strain vs. time plot (middle left panel). Approximately 1.5 cardiac signals are shown in the example shown in FIG. 2.

Step S120, which includes dynamically determining a time window from the defining signal, functions to determine a time window from an easily monitored signal (the defining signal) that can be used to process a second signal (e.g., tissue state ultrasound signal). Preferably, the time window is identified from the physiological signal, such as signals corresponding to heart electrical activity, heart rate, blood pressure, respiratory activity, respiration rate, brain waves, reflex reactions, perspiration rate, or any other suitable physiological signal. The duration of a time window is preferably defined by boundary markers. A time window is preferably dynamically determined by actively identifying boundary markers as the defining signal is measured. In other words, the time window is determined in real-time. Boundary markers are preferably any aspects of the defining signal that can be used to infer the timing of the defining window or of the tissue state. A boundary marker may be continuous (adjusted over a period of time) or discrete (a single event in time). There is preferably a start boundary marker that indicates that a time window should start and a stop boundary marker that indicates that a time window should stop. Alternatively, there could be any number of boundary markers. A single boundary marker may only be necessary if the boundary marker itself defines a time window (such as when detecting a signal pattern). There may alternatively be multiple boundary markers such as if a time window is defined for the occurrence of a particular number of events. The boundary markers are preferably trigger events, which are basic conditions of a signal defined by a rule. The trigger event may be a signal edge trigger (rising edge or falling edge), a signal level, a frequency threshold, amplitude threshold, a timing condition (e.g., within a range for an amount of time), and/or any suitable signal condition. The boundary marker may alternatively be identified by pattern recognition. The boundary markers are preferably positioned to capture a cardiac phase (or portion) of the cardiac cycle, such as the ventricular systole. The defining signal is preferably correlated to particular signal patterns that serve as indicators of an event. Alternatively, the time window processing may include low pass filtering, or any other suitable signal processing. As exemplified in FIG. 2, the electrocardiogram (ECG) signal may be used to determine the state of the cardiac cycle and define a time window for strain rate integration. Ventricular systole is preferably a part of the heart cycle that is of interest because of the amount of motion and resulting strain in the heart tissue. The QRS complex of an ECG is an indicator of the beginning of ventricular systole. So, in one example, a time window may have a start trigger event on the rising edge between Q and R. The time window may have an end trigger event after the detection of a T wave, at the start of the next QRS complex, or at any suitable time.

As an additional alternative, a plurality of time windows type may be determined from the defining signal. The plurality of time windows are preferably of varying types. A time window type is a time window with a set of boundary markers. These boundary markers are preferably set to target particular portions of a defining signal. The boundary markers are preferably set at particular phases of a cardiac cycle such as to capture the ventricular systole. The plurality of time window types would allow for specific segments of a defining signal to be detected, and possibly processed according to particular rules. For example, an ECG may have three types of time windows: one time window defining the P wave, another the QRS complex, and a third for the T wave.

Additionally, the step S120 may include predicting a time window from time window history S122, which functions to preemptively calculate the parameters of a time window. The predicted time window is preferably calculated from averaging previous time windows (of the same type). The predicted time window history may alternatively be preset according to outside results (e.g., data collected from a laboratory). The predicted time window may alternatively use pattern matching to identify indicators that relate to the length of a time window. The window prediction may be used in situations where a processing operation needs to know the duration of a time window during the processing. In this way processing of the instantaneous tissue state signal captured in the time window can undergo processing before the time window has ended. The predicted time window may additionally be used as an error check. If the current time window differs significantly from the predicted time window alternative actions may be taken such as ignoring or discarding data. The predicted time window may additionally be used to correct or modify a current time window.

Step S130, which includes processing the instantaneous tissue state signal captured in the determined time window, functions to extract data from the instantaneous tissue state signal according to a defined time window and to analyze the data. The processing preferably includes integrating or accumulating the tissue state measurements acquired within the determined time window. The integration and/or accumulation are preferably performed in real-time to enable real-time image display. As shown in FIG. 2, the strain rate is integrated over the time window determined from the ECG signal. Although the plots shown in FIG. 2 only show data from the region of interest, the accumulation and/or integration is preferably performed for all samples of the strain rate image to form the strain image. The processing may alternatively use the time window to convolve or correlate the time window of the tissue state with other signal patterns to identify signal patterns. The processing could alternatively be any suitable process. In the alternative where multiple window types are determined, the processing operation may be determined by the type of time window. For example, a time window associated with the QRS complex may undergo a certain time window processing while the time window associated with the T wave would undergo a second time window processing. The processing may alternatively use a weighting factor determined from the time window: either the type of time window, the time within a time window, or according to any suitable aspect of a time window.

As an additional step, the method may include outputting an instantaneous tissue state signal (e.g., ultrasound imaging signal) with processed time windows S140, which functions to produce the transformed tissue state signal. Preferably, the whole signal is transformed as a plurality of time windows define the whole signal. But alternatively only a portion of the ultrasound imaging signal may have been processed such as if the time window is only defined over the systole portion of the heart cycle. Step S140 preferably includes displaying the processed instantaneous tissue state signal at the time of data acquisition. There is preferably minimal delay between the collection of data and the displaying of the results of the processing, thus enabling real-time decisions. Here minimal delay is understood to be less than one second. The instantaneous tissue state signal is additionally simultaneously displayed with the processed instantaneous tissue signal. The display may be a graphical representation of the data (e.g., a graph), an image or series of images, or may alternatively be a number value. In some situations where the method is used to detect discrete events, alerts or status messages may alternatively be displayed.

An alternative embodiment preferably implements the above method in a computer-readable medium storing computer-readable instructions. The instructions are preferably executed by computer-executable components for processing a real-time ultrasound signal within a time window. The computer-readable medium may be stored on any suitable computer readable media such as RAMs, ROMs, flash memory, EEPROMs, optical devices (CD or DVD), hard drives, floppy drives, or any suitable device. The computer-executable component is preferably a processor but the instructions may alternatively or additionally be executed by any suitable dedicated hardware device.

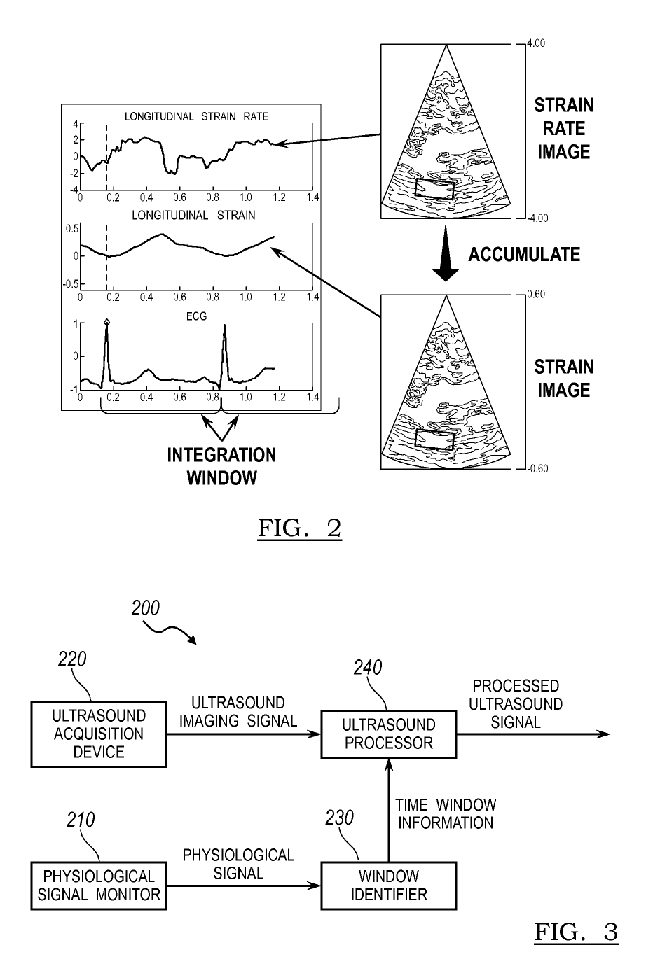

As shown in FIG. 3, a system 200 of the preferred embodiment includes a physiological signal monitor 210, an ultrasound acquisition device 220, a window identifier 230, and an ultrasound processor 240. The system 200 functions to acquire a physiological signal to determine a time window and process an ultrasound signal based on the time window. The system is preferably used to implement the above method. The physiological signal monitor 210 is preferably an electrocardiograph (ECG), but may alternatively be any suitable device used to measure a defining signal as described above. The physiological signal monitor 210 is preferably in electrical communication with the window identifier 230. The window identifier 230 preferably uses the physiological signal to determine a time window specified by set boundary markers. The window identifier 230 is preferably an edge detector capable of detecting rising and/or falling edges or alternatively, detect signal level, slope, frequency, amplitude, and/or any suitable signal characteristics. The window identifier 230 may alternatively be a pattern recognition processor or any suitable device that determines a time window. In one variation, the window identifier 230 preferably identifies a portion of a signal and more preferably identifies different cardiac phases such as the ventricular systole portion of a heart cycle. The ultrasound acquisition device 220 preferably obtains an ultrasound signal and more preferably an ultrasound image signal. The ultrasound acquisition device 220 preferably obtains an ultrasound imaging signal and, more preferably, a strain rate signal. The ultrasound acquisition device 220 may alternatively directly measure tissue data using an ultrasound transducer or be any suitable device for measuring a tissue state signal. The ultrasound acquisition device 220 is preferably in electronic communication with the ultrasound processor 140. The ultrasound processor 140 preferably processes the ultrasound imaging signal within the time window. The time window processed signal is preferably output in real-time. Any suitable processing operation may be performed. In one preferred embodiment, the ultrasound imaging signal is accumulated within the time window. In one example, the physiological signal monitor 210 is an ECG device and the ultrasound acquisition device 220 provides strain rate measurements. The window identifier 230 preferably identifies a time window defined by a heart cycle measured in the ECG. The ultrasound processor 240 then accumulates the strain rate measurement within the time window to output an accumulated strain measurement for a time window. The system 200 may additionally or alternatively be any suitable system that implements the above method.

As a person skilled in the art will recognize from the previous detailed description and from the figures and claims, modifications and changes can be made to the preferred embodiments of the invention without departing from the scope of this invention defined in the following claims.

We claim:

1. A method for processing a real-time ultrasound signal within a time window comprising the steps of:
measuring a physiological signal and an instantaneous ultrasound imaging signal;
identifying boundary markers in the physiological signal as the physiological signal is measured, wherein the boundary markers define a time window;
processing the ultrasound imaging signal captured in the time window; and
outputting an instantaneous ultrasound imaging signal with processed time windows.

2. The method of claim 1, wherein the step of identifying the boundary markers includes identifying a trigger event.

3. The method of claim 2, wherein the trigger event is identified by a signal edge detector.

4. The method of claim 2, wherein the step of identifying the boundary markers further includes identifying a start boundary marker and a stop boundary marker.

5. The method of claim 2, further comprising determining a plurality of time windows of varying types, each type having a set of boundary markers.

6. The method of claim 5, wherein the step of processing includes processing the ultrasound imaging signal according to the type of time window.

7. The method of claim 1, wherein the physiological signal is an electrocardiogram (ECG).

8. The method of claim 7, wherein the instantaneous measurement of the ultrasound imaging signal is a tissue velocity measurement.

9. The method of claim 7, wherein the instantaneous measurement of the ultrasound imaging signal is a strain rate measurement.

10. The method of claim 9, wherein the step of measuring the ultrasound imaging signal includes processing acquired ultrasound data to calculate the strain rate measurement.

11. The method of claim 9, wherein the strain rate measurement is at a frame rate of at least 100 Hz.

12. The method of claim 9, wherein the step of processing of the ultrasound imaging signal includes accumulating the ultrasound imaging signal over the time window.

13. The method of claim 12, wherein the time window has boundary markers positioned to capture a cardiac phase of a heart cycle.

14. The method of claim 1, wherein properties of a time window are predicted from a time window history.

15. The method of claim 14, including modifying a time window when the determined time window and a predicted time window differ more than a predetermined threshold.

16. A method for processing a real-time ultrasound signal within a time window comprising the steps of:
measuring an electrocardiogram (ECG) and identifying the ventricular systole portion of a heart cycle;
measuring the strain rate of at least a portion of the heart;
dynamically determining a time window from the ventricular systole portion;
accumulating the strain rate measurement captured in the time window; and
outputting a strain measurement.

17. A system for processing a real-time ultrasound signal within a time window comprising:
a physiological signal monitor that measures a physiological signal;
an ultrasound acquisition device that acquires an ultrasound signal;
a window identifier that uses the physiological signal to identify boundary markers of the output of the physiological signal monitor, wherein the boundary markers define a time window; and
an ultrasound processor that processes the ultrasound signal within a time window and outputs a time window processed ultrasound signal.

18. The system of claim 17, wherein the physiological signal monitor is an electrocardiogram (ECG).

19. The system of claim 18, wherein the window identifier is a signal edge detector.

20. The system of claim 19, wherein the window identifier is set to identify a cardiac phase of a heart cycle.

21. The system of claim 18, wherein the ultrasound signal is a measurement of the strain rate of at least a portion of a heart, and the ultrasound processor is an accumulator that outputs the accumulated strain during a time window.
